(19) United States
(12) Patent Application Publication
(10) Pub. No.: US 2019/0003935 A1
(43) Pub. Date: Jan. 3, 2019

(54) FILTRATION DEVICE WITH REMOVABLE PROTECTIVE MEMBER

(71) Applicant: BOSTON SCIENTIFIC SCIMED, INC., Maple Grove, MN (US)

(72) Inventors: JOSHUA CLARK, Spencer, IN (US); GENE T. STORBECK, Franklin, MA (US); SHANNON SMITH KENWOOD, Hopkinton, MA (US); CHRISTOPHER A. BENNING, Hopkinton, MA (US); SEAN CURRAN, Bourne, MA (US); SARAH JEAN HEIGHTON, Clinton, MA (US)

(73) Assignee: BOSTON SCIENTIFIC SCIMED, INC., Maple Grove, MN (US)

(21) Appl. No.: 16/019,078

(22) Filed: Jun. 26, 2018

Related U.S. Application Data
(60) Provisional application No. 62/527,447, filed on Jun. 30, 2017.

(51) Int. Cl. G01N 1/31; A61B 10/02; B01L 3/00; G01N 1/40; G01N 33/50

(52) U.S. Cl. CPC G01N 1/31; A61B 10/0283; B01L 3/5021; C12M 47/02; G01N 33/5088; B01L 2300/0618; B01L 2300/0681; G01N 1/4077

(57) ABSTRACT

A cell filtration assembly adapted to capture cells from a biological sample during centrifugation includes a filter support member including a sample well and a filter membrane that spans the sample well. In some cases, the filter support member and/or the filter membrane may be adapted to be sectioned. In some cases, a protective element may be disposed over at least a portion of the filter support member in order to protect the filter support member from tissue processing reagents during processing of the cells captured from the biological sample.

### CROSS-REFERENCE TO RELATED APPLICATIONS

This application claims priority under 35 U.S.C. § 119 to U.S. Provisional Application Ser. No. 62/527,447, filed Jun. 30, 3017, the entirety of which is incorporated herein by reference.

### TECHNICAL FIELD

The present disclosure pertains to medical devices, and methods for manufacturing medical devices. More particularly, the present disclosure pertains to filtration devices usable in processing biological samples.

### BACKGROUND

A wide variety of medical devices have been developed for medical use, for example for collecting and/or processing biological samples. Some of these devices include filtration devices.

### BRIEF SUMMARY

This disclosure provides design, material, manufacturing method, and use alternatives for medical devices. An example medical device includes a cell filtration assembly that is adapted to capture cells from a biological sample during centrifugation. The cell filtration assembly includes a filter support member including a sample well and a filter membrane spanning the sample well, where the filter support member and/or the filter membrane adapted to be sectioned. A protective element is disposed over at least a portion of the filter support member and is adapted to protect the filter support member from tissue processing reagents during processing of the cells captured from the biological sample.

A cell filtration assembly is disclosed. The cell filtration assembly comprises: a filter support member including a sample well; a filter membrane extending across the sample well; the filter support member and/or the filter membrane configured to be sectioned; and a protective element disposed over at least a portion of the filter support member, the protective element configured to protect the filter support member from tissue processing reagents during processing of the cells captured from the biological sample.

Alternatively or additionally to any of the embodiments above, the protective element is adapted to be removed prior to sectioning.

Alternatively or additionally to any of the embodiments above, the protective element includes a removable shell.

Alternatively or additionally to any of the embodiments above, the protective element is adapted to be sectioned while disposed over at least a portion of the filter support member.

Alternatively or additionally to any of the embodiments above, the protective element includes a polymeric coating.

Alternatively or additionally to any of the embodiments above, the protective element includes Parylene.

Alternatively or additionally to any of the embodiments above, the filter support member includes a material that is at least partially miscible in xylene.

Alternatively or additionally to any of the embodiments above, the filter support member includes a wax.

Alternatively or additionally to any of the embodiments above, the filter support member includes paraffin.

Another example medical device is a cell filtration assembly that is adapted to capture cells from a biological sample. The cell filtration assembly includes a sectionable base member and a sample well that is defined within the sectionable base member. A porous surface defines a lower portion of the sample well and is adapted to capture cells from the biological sample while permitting fluids to pass therethrough during centrifugation. A protective polymeric coating is disposed over at least a portion of the sectionable base member and protects the sectionable base member against tissue processing reagents. The protective polymer coating is sectionable.

Alternatively or additionally to any of the embodiments above, the protective polymer coating includes Parylene.

Alternatively or additionally to any of the embodiments above, the sectionable base member includes a material that is at least partially miscible in xylene.

Alternatively or additionally to any of the embodiments above, the sectionable base member includes a wax.

Alternatively or additionally to any of the embodiments above, the sectionable base member includes paraffin.

Alternatively or additionally to any of the embodiments above, the porous surface has an average pore size of about 0.1 to 50 micrometers.

Alternatively or additionally to any of the embodiments above, the porous surface has an average pore size of about 2 to 10 micrometers.

Another example medical device is a cell filtration assembly that is adapted to capture cells from a biological sample. The cell filtration assembly includes a sectionable base member, a sample well defined within the sectionable base member and a porous surface that defines a lower portion of the sample well and is adapted to capture cells from the biological sample while permitting fluids to pass therethrough during centrifugation. A protective shell is disposed over at least a portion of the sectionable base member and protects the sectionable base member against tissue processing reagents. The protective shell is removable prior to sectioning the sectionable base member.

Alternatively or additionally to any of the embodiments above, the cell filtration assembly further includes a protective cap that protects at least some portions of the sectionable base member that are not protected by the protective shell.

Alternatively or additionally to any of the embodiments above, the cell filtration assembly further includes a protective membrane that is securable to a lower surface of the sectionable base member to protect the lower surface of the sectionable base member against tissue processing reagents.

Alternatively or additionally to any of the embodiments above, the sectionable base member includes paraffin.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic illustration of a cell filtration assembly in accordance with the disclosure;

FIG. 2 is a cross-sectional view of the cell filtration assembly of FIG. 1, taken along line 2-2;

FIG. 3 is a schematic cross-sectional view of a cell filtration assembly in accordance with the disclosure;

FIG. 4 is a schematic cross-sectional view of a cell filtration assembly in accordance with the disclosure;

FIG. 5 is a schematic cross-sectional view of a cell filtration assembly in accordance with the disclosure;

FIG. 6 is a perspective view of a cell filtration assembly in accordance with the disclosure;

FIG. 7 is a schematic cross-sectional view of a cell filtration assembly in accordance with the disclosure;

FIGS. 8 through 10 illustrate use of the cell filtration assembly of FIG. 7; and

FIGS. 11A through 11C are illustrations of experimental results.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

There are a number of methods for the collection of biological samples by a biopsy and/or other surgical processes. Such processes typically result in a tissue sample that can be routinely processed for pathological analysis. In some endoscopic procedures such as those where a fine needle aspiration device is utilized, the sample that is collected includes loose cells and fluids. Prior to tissue processing and/or analysis, additional steps may be necessary to gather the desired cells/tissue and allow the cells/tissue to be further processed. Disclosed herein are devices and methods that allow cells/tissue to be efficiently processed and/or analyzed including cells/tissue gathered by fine needle aspiration devices and/or other devices that collect cells/tissue along with fluids. In some cases, for example, the devices and methods described herein may be used in processing cells, tissue or other biological samples obtained using other techniques as well.

In some cases, a biological sample including cells or other tissue within a fluid may be subjected to centrifugation in order to collect the cells or other tissue on a substrate for subsequent processing and examination while eliminating the fluid that previously carried the cells or other tissue. Once the cells are captured on a substrate such as but not limited to a filter membrane, they may be treated with other reagents, fixing agents, and the like, during tissue processing. The cells may subsequently be embedded in a medium, such as but not limited to a wax such as paraffin wax. In some cases, the cells embedded in a medium may be referred to as a cell block. The cell block may subsequently be sectioned into thin slices for mounting on a glass slide for analysis on a microscope, for example, or sliced from the cell block for other analytical processes. For example, visualization of the cells and the extracellular environment can provide information to determine whether the tissue collected is benign or malignant. Alternatively, the slices provide cellular material (DNA, RNA, proteins) for microcellular analysis.

In some cases, a biological sample may be initially processed by placing the biological sample, which typically includes cells or other tissue of interest, within a fluid, into a centrifuge tube and spinning or otherwise centrifuging the biological sample in order to capture the cells or other tissue of interest on a substrate such as a filter membrane while driving off the extraneous fluid. In some cases, the centrifuge tube may include a fixative, which refers to a compound that helps to preserve the cells or other tissue of interest. Illustrative but non-limiting examples of suitable fixatives include formalin, ethanol and methanol. In some cases, saline may be included as a holding solution. Another example is RPMI medium, or Rosewell Park Memorial Institute medium, which is a medium used in cell culture and tissue culture. In some cases, a centrifuge tube may have a volume ranging from about 15 milliliters (ml) to about 50 ml, although this is merely illustrative. In some instances, the centrifuge tube may accommodate a filter membrane upon which the cells or other tissue of interest may be collected, as will be discussed subsequently. The centrifuge tube may be spun in a standard centrifuge at speeds that subject the contents of the centrifuge tube to relative centrifugal forces (RCF) of between about 200 to about 1800 RCF.

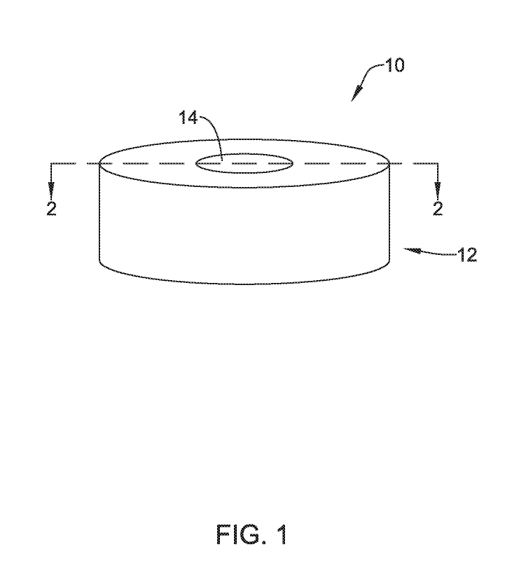

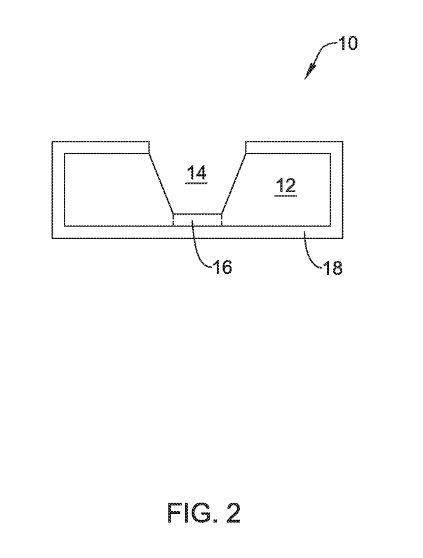

FIG. 1 is a schematic perspective view of an illustrative cell filtration assembly 10 that may be considered as being adapted to capture cells from a biological cell during centrifugation while FIG. 2 is a cross-sectional view along line 2-2 of FIG. 1. The cell filtration assembly 10 includes a filter support member 12. In some cases, the filter support member 12 may be considered as being a sectionable base member. The filter support member 12 includes or defines a sample well 14. In some cases, the filter support member 12 may be made of or otherwise include a material that is at least partially miscible in tissue processing reagents such as xylene (dimethylbenzene). For example, the filter support member 12 may be made of or otherwise include a wax such as but not limited to paraffin, which is a white or colorless soft solid derivable from petroleum, coal or oil shale, that generally includes a mixture of hydrocarbon molecules containing between twenty and forty carbon atoms.

A filter membrane 16 extends across a lower end (in the illustrated orientation) of the sample well 14. It will be appreciated that the filter support member 12 and/or the filter membrane 16 may be configured to be sectioned. In some cases, the filter membrane 16 may be considered as being a porous surface that defines a lower portion of the sample well 14. A protective element 18 may be disposed over at least a portion of the filter support member 12 and may, for example, be adapted to protect the filter support member 12 or at least portions thereof from tissue processing reagents during processing of the cells captured from the biological sample. In some cases, the filter membrane 16 may include a porous material with openings sized to allow the desired cells/tissue to be collected thereon while allowing fluids to pass therethrough. For example, the filter membrane 16 have pores that are about 0.1-50 micrometers, or about 1-20 micrometers, or about 2-10 micrometers, or about 5 micrometers, or smaller than about 10 micrometers, or smaller than about 5 micrometers, or the like.

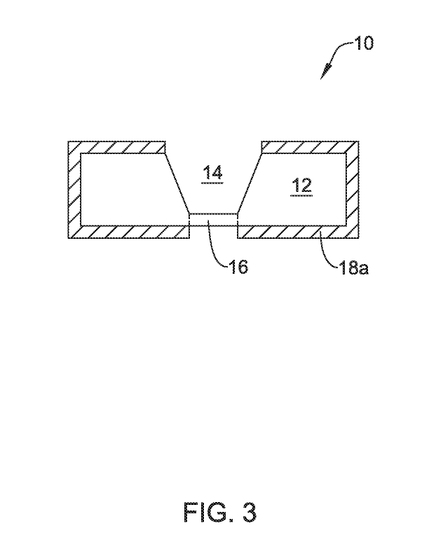

In some cases, the protective element 18 may be configured to remain disposed on at least a portion of the filter support member 12, and may in fact be configured to subsequently be sectioned along with the filter support member 12 and/or the filter membrane 16 (and the cells or other tissue of interest disposed on the filter membrane 16). FIG. 3 is a schematic cross-section of FIG. 1, illustrating the protective element 18 as being a polymeric coating 18a. The polymeric coating 18a may cover all of the filter support member 12, or in some cases the polymeric coating 18a may only cover a portion of the filter support member 12. The polymeric coating 18a may be formed via a dipping process, a spraying process or any other suitable deposition process, and may be any usable coating thickness. In some cases, for example, the polymeric coating 18a may be formed of or may otherwise include a crystalline wax, polyacrylic acid, cyanoacrylate, phenol formaldehyde, urethanes such as two part urethanes, diglycidyl ether, fluorosilicone, and the like. In some cases, the polymeric coating 18a may include Parylene. Parylene refers to a variety of chemical vapor deposited poly(p-xylylene) polymers. Parylene C, for example, is a popular polymer for a variety of applications due to its combination of barrier properties, cost and other processing advantages.

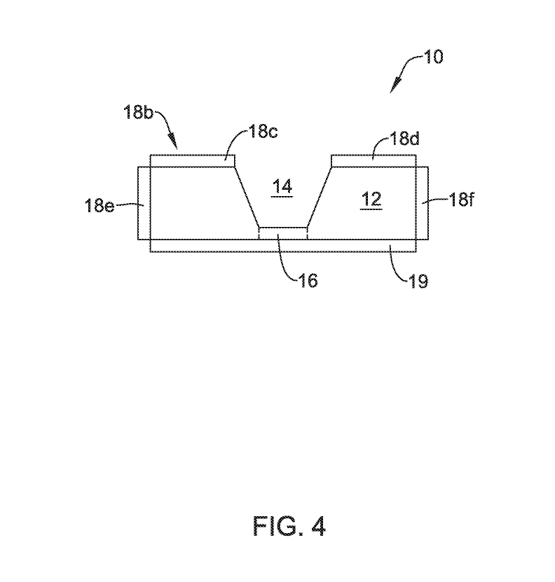

In some cases, the protective element 18 (FIG. 2) may not be easily sectionable, and may instead be configured to be removed prior to sectioning. FIG. 4 is a schematic cross-sectional view of FIG. 1, illustrating the protective element 18 as being a protective shell 18b. In some cases, the protective shell 18b may be formed of distinct segments 18c, 18d, 18e and 18f (as illustrated). In some cases, the protective shell 18b may instead be formed as a unitary structure, as long as the protective shell 18b can be easily removed after tissue processing and before sectioning. The protective shell 18b may be formed of any suitable material that resists tissue processing reagents such as but not limited to xylene. Particular examples include ABS (acrylonitrile butadiene styrene), Nylon, PEEK (polyetheretherketone), PTFE (polytetrafluoroethylene), PVDF (polyvinylidene fluoride), PP (polypropylene) and various metals. In some cases, a thin membrane 19 may extend across a lower surface of the filter support member 12 to protect the lower surface against tissue processing reagents.

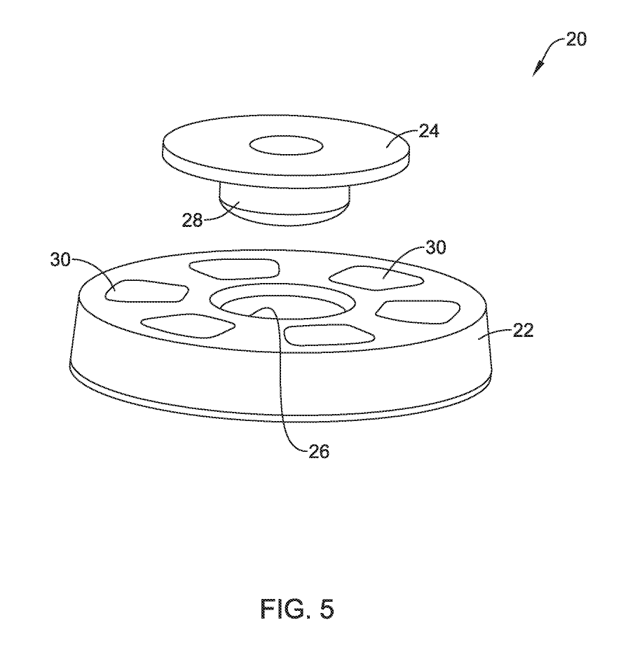

FIG. 5 is a perspective view of an illustrative cell filtration assembly 20 that may be used in capturing cells or other tissue of interest from a biological sample. In some cases, the cell filtration assembly 20 includes a body portion 22 and a cap 24 that may be secured relative to the body portion 22 during various processing procedures. It can be seen that the body portion 22 defines a sample well 26, and that the cap 24 includes an annular extension 28 that may be sized to fit within the sample well 26. In some cases, as illustrated, the body portion 22 includes voids 30 that facilitate grasping the cell filtration assembly 20 with tweezers, for example. In some cases, the voids 30 allow for embedding wax to fully surround the body portion 22 and help to hold it together during sectioning. In some cases, a particular pattern of the voids 30 may also facilitate sectioning.

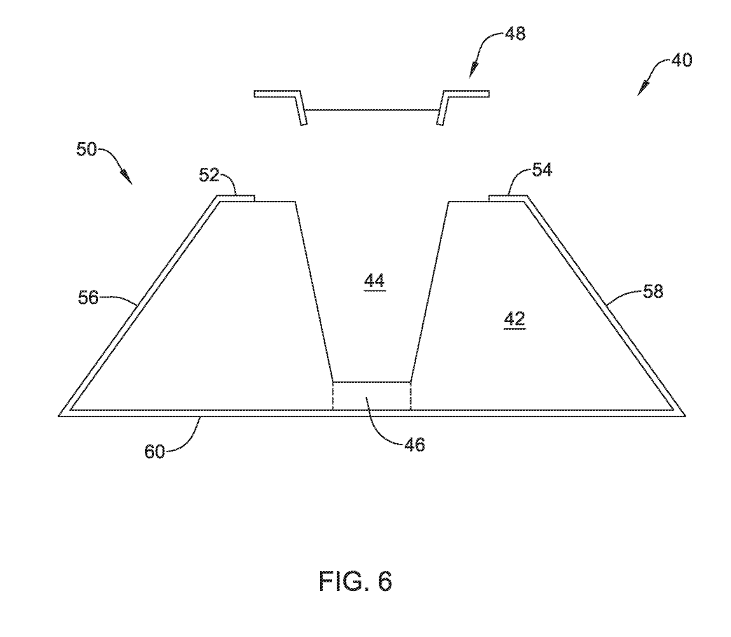

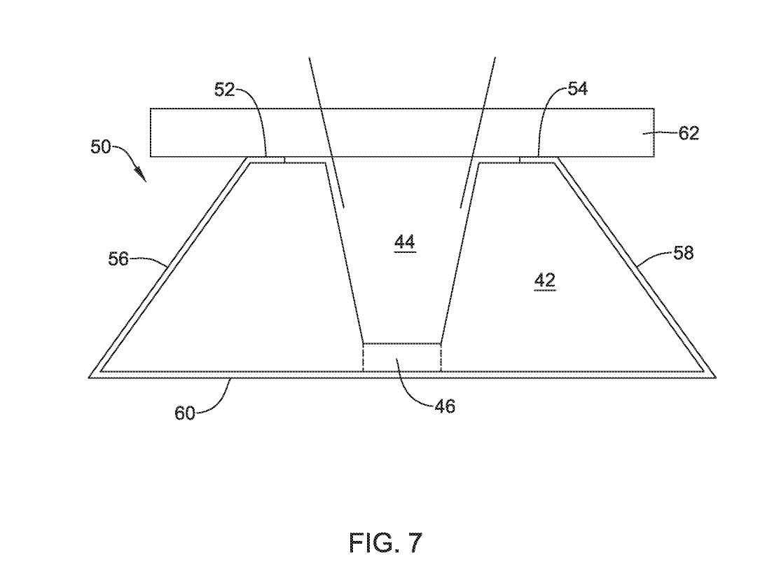

FIG. 6 is a schematic cross-sectional view of an illustrative cell filtration assembly 40 that may be considered as being an example of the cell filtration assembly 10 shown in FIG. 4, including the protective shell 18b. As illustrated, the cell filtration assembly 40 includes a filter support member 42 defining a sample well 44. A lower member 46 extends across a lower portion of the sample well 44. In some cases, the lower member 46 may include or otherwise provide a filter membrane. In some cases, the lower member 46 may be impervious to fluid flow. A cap 48 is configured to fit along the top of the filter support member 42 to help protect the filter support member 42 against tissue processing reagents such as but not limited to xylene. The cell filtration assembly 40 includes a protective shell 50 that in some cases may include distinct segments or portions 52, 54, 56 and 58. In some cases, the protective shell 50 may be formed having fewer distinct segments or portions. In some cases, a thin membrane 60 may extend across the bottom (in the illustrated orientation) of the filter support member 42. FIGS. 7 through 10 illustrate use of the cell filtration assembly 40 in capturing a cell sample.

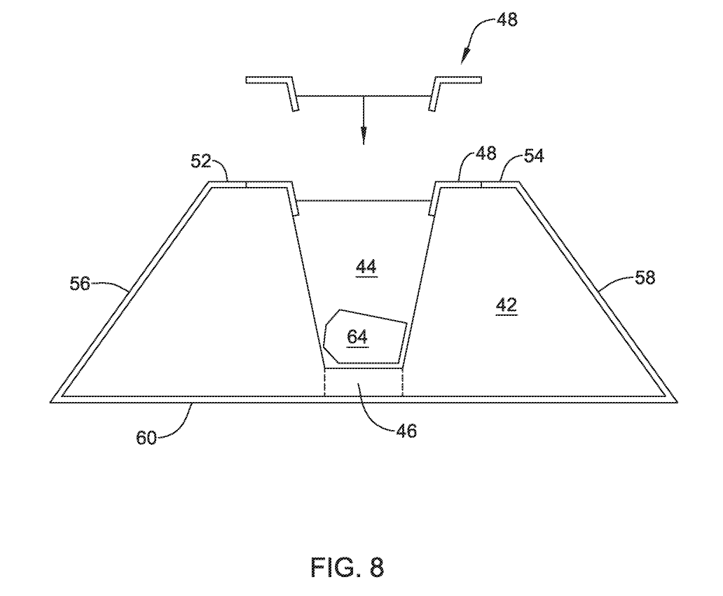

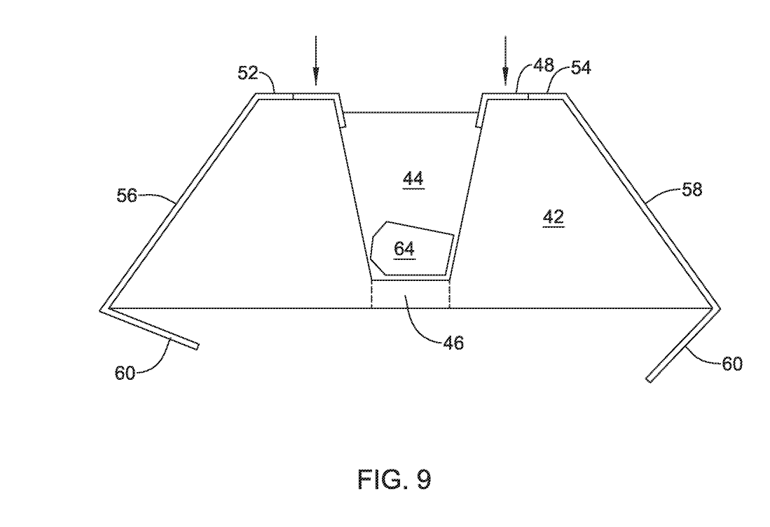

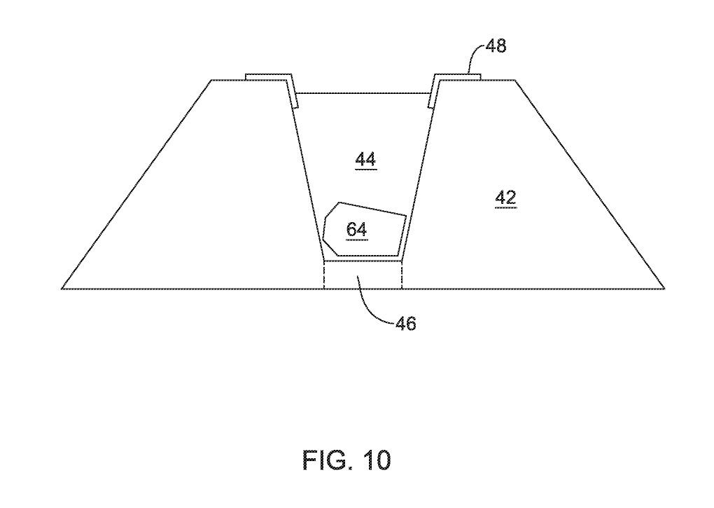

In FIG. 7, the cell filtration assembly 40 has been secured relative to an inner sample tube 62 and is subject to centrifugation. This causes the cells or other tissue of interest to move downward into the sample well 44 and be captured on the lower member 46, thereby forming a sample 64. The cell filtration assembly 40 may then be removed from the centrifuge and capped with the cap 48, as seen in FIG. 8, and then subjected to additional tissue processing procedures. After tissue processing, and with reference to FIG. 9, the filter support member 42 may be pushed downward through the thin membrane 60, releasing the filter support member 42 and the sample 64 from the protective shell 50. The results are shown in FIG. 10. After this, the cap 48 may be removed, and the cell filtration assembly 40 may subsequently be embedded in paraffin and cut into slices for diagnosis and ancillary tests. In some cases, the cap 48 may remain in place in order to prevent cell loss.

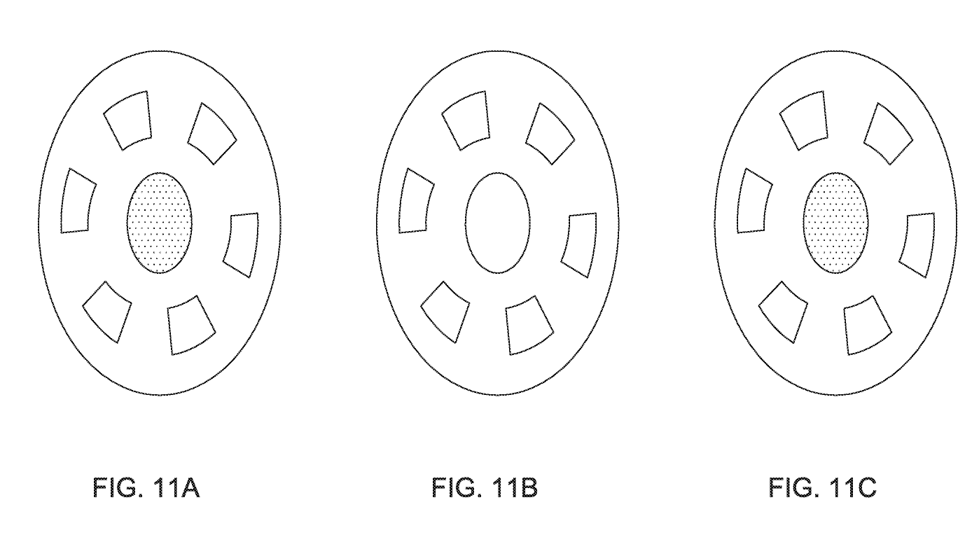

FIGS. 11A through 11C illustrate the results of using a protective coating or element to help protect the cell filtration assembly against tissue processing reagents such as xylene. FIG. 11A provides a view of the base portion 22 (FIG. 5) of a cell filtration assembly. This illustrates the baseline, before exposure to xylene. FIG. 11B shows the results of exposing the base portion 22 to xylene at 45° C. for a period of three hours. As can be seen, the thin center portion (layer of paraffin wax) can be seen as having been completed eroded away. In comparison, FIG. 11C shows the results of exposing the base portion 22, coated with Parylene, to xylene at 45° C. for a period of three hours. As can be seen, the Parylene coating results in visibly less erosion and degradation.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A cell filtration assembly comprising:
a filter support member including a sample well;
a filter membrane extending across the sample well;
the filter support member and/or the filter membrane configured to be sectioned; and
a protective element disposed over at least a portion of the filter support member, the protective element configured to protect the filter support member from tissue processing reagents during processing of the cells captured from the biological sample.

2. The cell filtration assembly of claim 1, wherein the protective element is adapted to be removed prior to sectioning.

3. The cell filtration assembly of claim 2, wherein the protective element comprises a removable shell.

4. The cell filtration assembly of claim 1, wherein the protective element is adapted to be sectioned while disposed over at least a portion of the filter support member.

5. The cell filtration assembly of claim 4, wherein the protective element comprises a polymeric coating.

6. The cell filtration assembly of claim 4, wherein the protective element comprises Parylene.

7. The cell filtration assembly of claim 1, wherein the filter support member comprises a material that is at least partially miscible in xylene.

8. The cell filtration assembly of claim 1, wherein the filter support member comprises a wax.

9. The cell filtration assembly of claim 1, wherein the filter support member comprises paraffin.

10. A cell filtration assembly adapted to capture cells from a biological sample, the cell filtration assembly comprising:
a sectionable base member;
a sample well defined within the sectionable base member;
a porous surface defining a lower portion of the sample well, the porous surface adapted to capture cells from the biological sample while permitting fluids to pass therethrough during centrifugation; and
a protective polymeric coating disposed over at least a portion of the sectionable base member;
wherein the protective polymeric coating protects the sectionable base member against tissue processing reagents; and
wherein the protective polymer coating is sectionable.

11. The cell filtration assembly of claim 10, wherein the protective polymer coating comprises Parylene.

12. The cell filtration assembly of claim 10, wherein the sectionable base member comprises a material that is at least partially miscible in xylene.

13. The cell filtration assembly of claim 10, wherein the sectionable base member comprises a wax.

14. The cell filtration assembly of claim 10, wherein the sectionable base member comprises paraffin.

15. The cell filtration assembly of claim 10, wherein the porous surface has an average pore size of about 0.1 to 50 micrometers.

16. The cell filtration assembly of claim 10, wherein the porous surface has an average pore size of about 2 to 10 micrometers.

17. A cell filtration assembly adapted to capture cells from a biological sample, the cell filtration assembly comprising:
a sectionable base member;
a sample well defined within the sectionable base member;
a porous surface defining a lower portion of the sample well, the porous surface adapted to capture cells from the biological sample while permitting fluids to pass therethrough during centrifugation; and
a protective shell disposed over at least a portion of the sectionable base member;
wherein the protective shell protects the sectionable base member against tissue processing reagents; and
wherein the protective shell is removable prior to sectioning the sectionable base member.

18. The cell filtration assembly of claim 17, further comprising a protective cap that protects at least some portions of the sectionable base member that are not protected by the protective shell.

19. The cell filtration assembly of claim 17, further comprising a protective membrane that is securable to a lower surface of the sectionable base member to protect the lower surface of the sectionable base member against tissue processing reagents.

20. The cell filtration assembly of claim 17, wherein the sectionable base member comprises paraffin.
